(19) United States
(12) Patent Application Publication
(10) Pub. No.: US 2009/0112325 A1
(43) Pub. Date: Apr. 30, 2009

(54) FOOTPLATE MEMBER AND A METHOD FOR USE IN A VERTEBRAL BODY REPLACEMENT DEVICE

(75) Inventors: Daniel REFAI, Saint Louis, MO (US); Jeffrey A. FARRIS, Berne, IN (US)

Correspondence Address: HESLIN ROTHENBERG FARLEY & MESITI PC, 5 COLUMBIA CIRCLE, ALBANY, NY, 12203, US

(73) Assignee: BIOSPINE, LLC, Columbia City, IN (US)

(21) Appl. No.: 11/928,553

(22) Filed: Oct. 30, 2007

(51) Int. Cl. A61F 2/44

(52) U.S. Cl. 623/17.16; 623/17.15

(57) ABSTRACT

The footplate member includes an end surface, a sidewall that is attached to the end surface, and an orientation mechanism that is designed to align the footplate member in a certain position relative to at least one of the two end members of the vertebral body replacement device. The orientation mechanism includes a plurality of tabs located on an end wall of each end member and corresponding slots located along the edge of the sidewall, with the tabs and slots being sized and positioned to mate. The footplate member also includes a locking mechanism designed to couple the footplate member to an end member prior to the implantation of the vertebral body replacement device within a space within a spinal column. A method for assembling a vertebral body replacement device and a method for using a footplate member in a vertebral body replacement device is also disclosed.

### CROSS-REFERENCE TO RELATED APPLICATIONS

This application is related to concurrently filed and commonly owned U.S. Non-Provisional patent application Ser. No. ______, entitled “VERTEBRAL BODY REPLACEMENT DEVICE AND METHOD FOR USE TO MAINTAIN A SPACE BETWEEN TWO VERTEBRAL BODIES WITHIN A SPINE” by REFAI et al. (Attorney Docket No. 2890.002).

### TECHNICAL FIELD

The present invention relates generally to orthopaedic and neurosurgical implants used for insertion within the spine, and more specifically, but not exclusively, concerns devices implanted within the spinal column to replace a resected, fractured or diseased vertebral body and to maintain or reestablish proper spacing between the remaining adjacent vertebral bodies.

### BACKGROUND OF THE INVENTION

Damage or disease that affects the integral structure of a vertebral body within an individual's spinal column may lead to neurologic impairment with possible permanent damage to the spinal cord as well as improper neck and back alignment. Maintaining anatomic spacing within the spinal column is critical to ensuring continued functionality of the spinal cord and nerve roots and avoidance of long term serious neurological impairment.

Typically, spinal implants that are used as a spacer type of device have a fixed overall length and are implanted without the ability to adjust the degree of expansion or curvature. Recent developments of spinal spacers have resulted in devices that may be lengthened in vivo by rotary motion to match the space presented by the missing vertebral body. Problems that have been seen with these types of designs include post-placement migration attributable to the torsional forces applied to the implant during the lengthening process risking the patient to neurologic injury, the improper sizing of the implant relative to the presented clinical space, limited device access ports for height manipulation, and the lack of endplate angulation possibilities.

### SUMMARY OF THE INVENTION

Advancement of the state of spinal implants and the surgical management relating to the clinical presentation of missing or damaged vertebral bodies within an intact spinal column is believed desirable. The present invention satisfies the need for improvements to the vertebral space implant used to treat patients suffering from either diseased or damaged vertebral bodies by providing an in vivo adjustable vertebral body replacement device for use within a spinal column that eliminates torsional forces being applied at the implant vertebral body interface, maintains the desired optimized height, and offers 360 degrees of adjustment tool access for allowing lengthening and shortening of the device in vivo.

The present invention provides in one aspect, a footplate member for use in a vertebral body replacement device, the vertebral body replacement device includes a body member, a central rod and at least two end members with the central rod member being configured to be operatively associated within the body member and engage the at least two end members. The footplate member has an end surface with the end surface being configured to engage a bone surface upon implantation of the vertebral body replacement device and a sidewall that is attached to the end surface. The sidewall is configured to include an orientation mechanism that functions to align the footplate member in a certain position relative to each of the at least two end members.

The present invention provides in yet another aspect, a method for assembling a vertebral body replacement device. The method includes the step of obtaining a body member that is an elongate body having an inner wall and an outer wall and includes a first end receptacle, a second end receptacle and a longitudinal axis extending between the first end receptacle and the second end receptacle. The method also includes the step of obtaining a central rod member that has a first threaded portion, a second threaded portion and a central axis extending therebetween. The central rod member is configured to be operatively associated with the body member. The method includes the step of obtaining a first end member and a second end member with the first end member being configured to be positioned within the first end receptacle of the body member to threadingly engage the first threaded portion of the central rod member when the central rod member is operatively associated with the body member, and the second end member being configured to be positioned within the second end receptacle of the body member to threadingly engage the second threaded portion of the central rod member when the central rod member is operatively associated with the body member. The method includes the further step of obtaining at least one footplate member. The at least one footplate member has an end surface with the end surface being configured to engage a bone surface upon implantation of the vertebral body replacement device. The at least one footplate member also has a mating surface attached to the end surface with the mating surface being configured to have an adjustment mechanism. The adjustment mechanism functions to align the at least one footplate member in a certain position relative to the first end member and/or the second end member. The at least one footplate member also has a locking mechanism that is configured to securely couple the at least one footplate member to the first end member and/or the second end member, thus allowing the footplate member to be modular in function. The method also includes the step of placing the central rod member within a middle chamber of the body member and operatively associating the central rod member with the body member. The method may include the step of threadingly engaging the first threaded portion of the central rod member with the first end member and threadingly engaging the second threaded portion of the central rod member with the second end member. An additional step of the method may include employing the locking mechanism to connect at least one footplate member to the first end member and/or the second end member.

The present invention provides in another aspect, a method of using at least one footplate member in a vertebral body replacement device. The method includes the step of obtaining a vertebral body replacement device, the vertebral body replacement device has a body member, a central rod member including a first threaded portion and a second threaded portion. The central rod member is configured to be operatively associated within the body member and a first end member and a second end member. The first end member is configured to threadingly engage the first threaded portion of the central rod member and the second end member is configured to threadingly engage the second threaded portion of the central rod member. The method also includes the step of obtaining at least one footplate member that has an end surface and a mating surface that is attached to the end surface. The mating surface is configured to have an adjustment mechanism which functions to align the at least one footplate member in a certain position relative to the first end member and/or the second end member. The at least one footplate member also has a locking mechanism that is configured to securely couple the at least one footplate member to the first end member and/or the second end member. The method includes the further step of coupling the at least one footplate member to the first end member and/or the second end member with the locking mechanism securely connecting the at least one footplate member to the first end member and/or the second end member.

Further, additional features and advantages are realized through the techniques of the present invention. Other embodiments and aspects of the invention are described in detail herein and are considered a part of the claimed invention.

### BRIEF DESCRIPTION OF THE DRAWINGS

The subject matter which is regarded as the invention is particularly pointed out and distinctly claimed in the claims at the conclusion of the specification. The foregoing and other objects, features and advantages of the invention are apparent from the following detailed description taken in conjunction with the accompanying drawings in which:

FIG. 1 is a perspective, exploded view of one embodiment of a vertebral body replacement device, in accordance with an aspect of the present invention;

FIG. 2A is a cross-sectional, side elevational view of an end member of the vertebral body replacement device of FIG. 1 taken along line 2-2, showing an inner portion with a surrounding external wall, an internal wall and an end wall with the inner portion including a centrally oriented threaded housing element configured to engage a central rod member with the end wall being oriented normal relative to the external wall, in accordance with an aspect of the present invention;

FIG. 2B is a cross-sectional, side elevational view of an alternative embodiment of an end member, showing an inner portion with a surrounding external wall, an internal wall and an end wall with the inner portion including a centrally oriented threaded housing element configured to engage a central rod member with the end wall being oriented at an angle relative to the external wall, in accordance with an aspect of the present invention;

FIG. 3 is a cross-sectional, side elevational view of a body member of the vertebral body replacement device of FIG. 1 taken along line 3-3, showing two receptacle ends and internal threads for engaging a support ring, in accordance with an aspect of the present invention;

FIG. 4 is a side elevational view of a central rod member of the vertebral body replacement device of FIG. 1, in accordance with an aspect of the present invention;

FIG. 5 is a side elevational view of the assembled vertebral body replacement device of FIG. 1, showing a superiorly positioned end member and an inferiorly positioned end member extended away from the body member, in accordance with an aspect of the present invention;

FIG. 6 is a perspective view of the vertebral body replacement device of FIG. 1, with a tool inserted through a tool port hole and in operable position with the central rod member, in accordance with an aspect of the present invention;

FIG. 7 is a side elevational view of the vertebral body replacement device of FIG. 1, shown disposed within a space between two vertebral bodies within a spinal column prior to the translational movement of the superiorly positioned end member and the inferiorly positioned end member, in accordance with an aspect of the present invention;

FIG. 8 is a side elevational view of the vertebral body replacement device of FIG. 1, shown positioned between two vertebral bodies with the superiorly positioned end member and the inferiorly positioned end member extended to maintain a desired space within a spinal column, in accordance with an aspect of the present invention;

FIG. 9 is a perspective view of an alternative embodiment of a vertebral body replacement device, with a superiorly positioned, detachable footplate member and an inferiorly positioned, detachable footplate member shown prior to being coupled to the superiorly positioned end member and an inferiorly positioned end member, respectively, in accordance with an aspect of the present invention;

FIG. 10A is a side elevational view of a detachable footplate member of the vertebral body replacement device of FIG. 9, showing an end surface being oriented normal relative to a sidewall, in accordance with an aspect of the present invention; and

FIG. 10B is a side elevational view of an alternative embodiment of a detachable footplate member used with the vertebral body replacement device of FIG. 9, showing the end surface being oriented at an angle relative to the sidewall, in accordance with an aspect of the present invention.

### BEST MODE FOR CARRYING OUT THE INVENTION

Generally stated, disclosed herein is a vertebral body replacement device or vertebral spacer that typically includes a body member, a central rod member, a support ring, two end members and at least one footplate member. As used herein, the terms “vertebral body replacement device” and “vertebral spacer” may be used interchangeable as they essentially describe the same type of implant device. Further, described herein is a surgical method for using the vertebral body replacement device to maintain a space between two vertebral bodies within a patient suffering from a diseased or damaged spinal column.

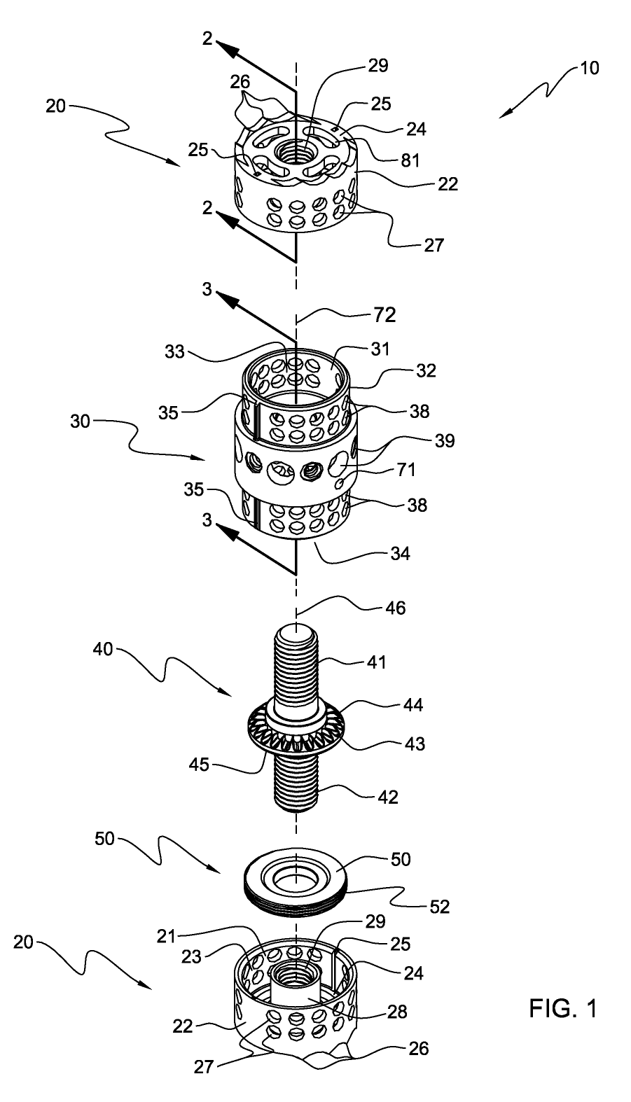

As depicted in FIG. 1, the general arrangement of a vertebral body replacement device 10, in accordance with an aspect of the present invention, includes a body member 30, at least two end members 20, a central rod member 40 and a support ring 50. In this detailed description and the following claims, the words proximal, distal, anterior, posterior, medial, lateral, superior and inferior are defined by their standard usage for indicating a particular part of a bone or prosthesis according to the relative disposition of the natural bone or directional terms of reference. For example, “proximal” means the portion of a prosthesis nearest the torso, while “distal” indicates the portion of the prosthesis farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure.

With reference to FIG. 1, vertebral body replacement device 10 includes body member 30, at least two end members 20 positioned superior and inferior relative to body member 30, a central rod member 40 for placement within body member 30 and support ring 50 that is configured to contact and secure central rod member 40 within body member 30.

Exhibited in FIG. 1, body member 30 also includes an inner wall 31 and an outer wall 32, at least one hole 38 extending from outer wall 32 through inner wall 31. Further, body member 30 has at least one anti-rotational rib 35 disposed on and extending for substantially the entire length of outer wall 32. At least one rib 35 is oriented in a superior to inferior direction relative to body member 30 and substantially parallel to a longitudinal axis 72 of body member 30. At least one hole 38 is used for the placement of bone graft or other biocompatible material that will facilitate bone fusion to occur in vivo following implantation of the device. It should be understood to those skilled in the art that body member 30 may be available to the operating surgeon in various outside diameter sizes and longitudinal lengths L (see FIG. 3). Having multiple sized body members 30 as part of an implant system allows the operating surgeon to use vertebral body replacement device 10 in various levels or segments of the spine (i.e., smaller sizes in the cervical spine, medium sizes in the thoracic spine and larger sizes in the lumbar spine).

As shown in the cross-sectional view of FIG. 3, body member 30 further includes a first or superiorly positioned end receptacle 33 and a second or inferiorly positioned end receptacle 34 with longitudinal axis 72 extending between these two structures within elongate body member 30. A middle chamber 36 is defined by inner wall 31 and is bound superiorly by first end receptacle 33 and inferiorly by second end receptacle 34. At least one tool port hole 39 extends into middle chamber 36 through outer wall 32 and inner wall 31. In addition, inner wall 31 of middle chamber 36 includes a set of internal threads 37 positioned in the bottom portion of middle chamber 36. Internal threads are sized and configured to threadingly engage the external threads 52 of support ring 50 (not shown). A ceiling surface 74 bounds the superior portion of middle chamber 36 with a centralized opening 75 positioned through ceiling surface 74. Although not shown, when vertebral body replacement device 10 is fully assembled and in use, central rod member 40 is operatively associated with body member 30 by being configured to allow for a superior threaded portion 41 of central rod member 40 to pass through centralized opening 75 resulting in a collar element 47 of central rod member 40 contacting ceiling surface 74. Following placement of superior threaded portion 41 of central rod member 40 through centralized opening 75, central rod member 40 is moveably secured within middle chamber 36 by threadingly coupling support ring 50 to internal threads 37 of middle chamber 36 resulting in a bearing surface 51 of support ring 50 making pressing contact with a support surface 45 of central rod member 40. Body member 30 further includes at least one locking pin hole 71 (as seen in FIG. 1) that passes through outer wall 32 and inner wall 31 into middle chamber 36. Although not shown, following final placement and adjustment of assembled vertebral body replacement device 10, a corresponding threaded pin or bolt may screw into at least one locking pin hole 71 resulting in central rod member 40 being secured in position, fixing the overall length of vertebral body replacement 10.

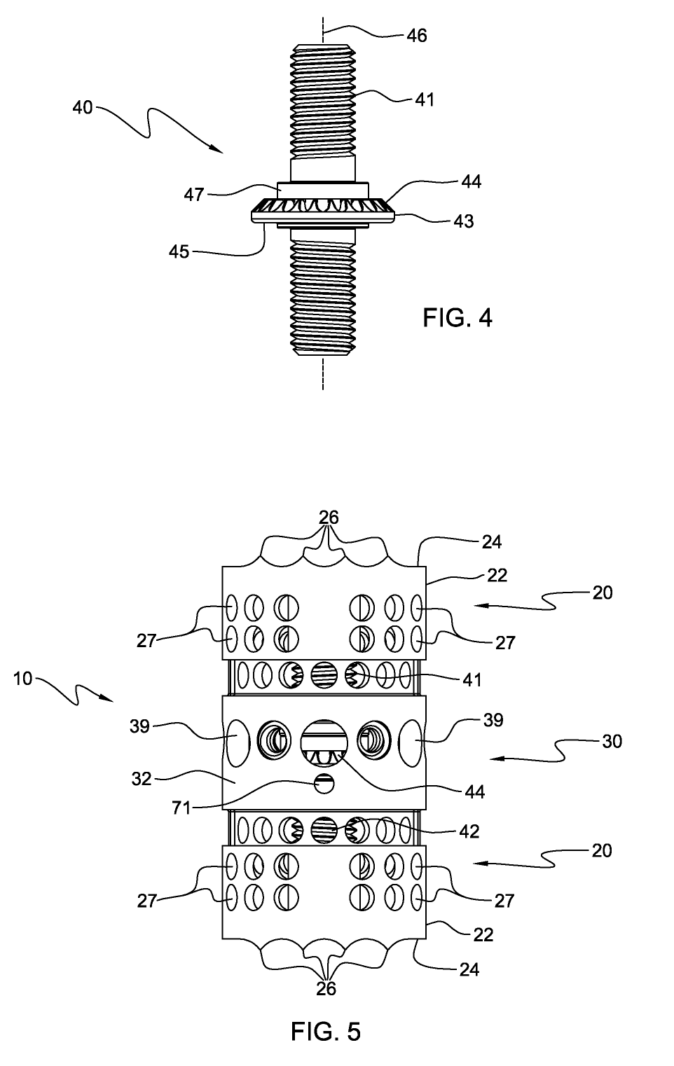

FIGS. 1 and 4 show central rod member 40 having first or superior threaded portion 41 and a second or inferior threaded portion 42 with the two threaded portions having opposing thread configurations. This means that when first threaded portion 41 is constructed with right-handed threads, second threaded portion 42 is constructed with left-handed threads. It should be understood to those skilled in the art that the vice-versa thread configuration is also contemplated. Central rod member 40 further includes a central axis 46 that passes from first threaded portion 41 to second threaded portion 42 with a gear wheel portion 43 being positioned intermediate first threaded portion 41 and second threaded portion 42. Gear wheel portion 43 is generally constructed with a toothed face surface 44, the plane of toothed face surface 44 being oriented substantially perpendicular to central axis 46. Collar element 47 is positioned adjacent to tooth face surface 44 to ensure proper external access of tooth face surface 44 within middle chamber 36 following assembly of vertebral body replacement device 10. Further, gear wheel portion 43 includes support surface 45 that is located on the inferior aspect or underside of gear wheel portion 43. Similar to that described for toothed wheel surface 44, the plane of support surface 45 is correspondingly oriented substantially perpendicular to central axis 46. As explained previously, support surface 45 will contact and slidingly articulate with bearing surface 51 of support ring 50 (see FIG. 1) when vertebral body replacement device 10 is assembled and in use. Gear wheel portion 43 is integral to central rod member 40 and is positioned so that when gear wheel portion 43 is moved about its rotational axis, first threaded portion 41 and second threaded portion 42 will also rotate because gear wheel portion 43 axis of rotation is coaxial with central axis 46.

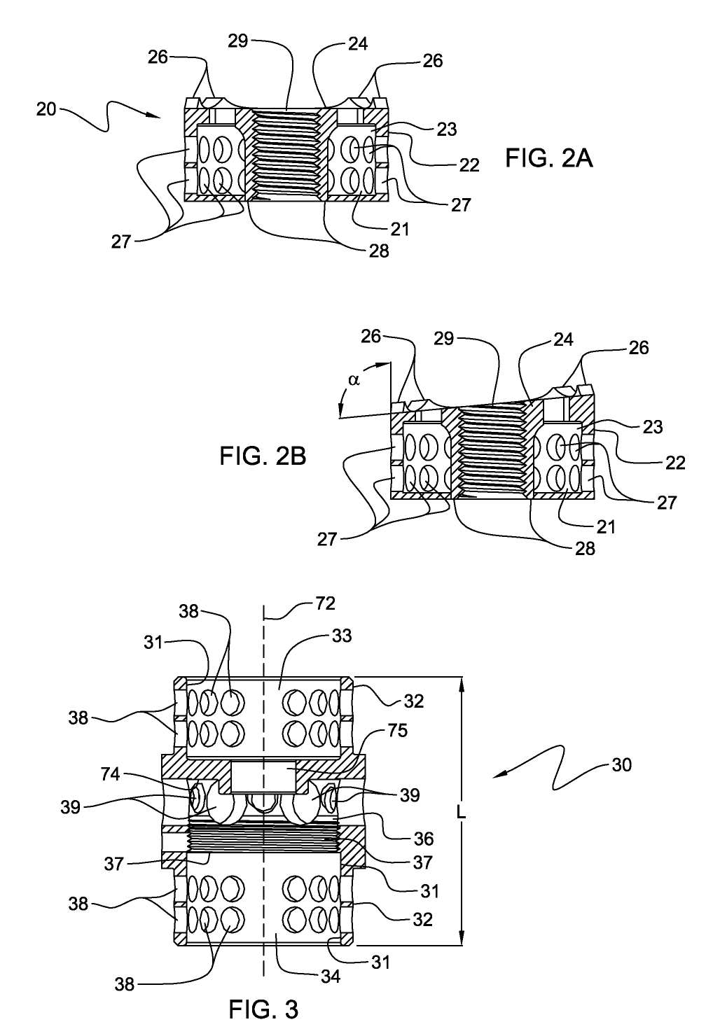

FIGS. 1, 2A and 2B depict end member 20. Vertebral body replacement device 10 includes in its construct at least two end members 20, with the first one end member 20 being positioned superiorly relative to body member 30 and the second end member 20 being positioned inferiorly relative to body member 30. In operation, superiorly positioned first end member 20 is aligned and concentric with first end receptacle 33 so that when first end member 20 moves relative to body member 30, an internal wall 23 of end member 20 is continuously positioned adjacent to outer wall 32 of first end receptacle 33. The same operational relationship occurs with inferiorly positioned second end member 20 as it will be aligned and concentric with second end receptacle 34 so that when second end member 20 moves relative to body member 30, internal wall 23 of end member 20 is continuously positioned adjacent to outer wall 32 of second end receptacle 34.

As seen in FIGS. 2A and 2B, end member includes an inner portion 21 that is bounded by internal wall 23 and a centrally positioned threaded housing element 28. Threaded housing element 28 is constructed with internal threads 29 that may extend the full length of threaded housing element 28. Internal threads 29 are configured to correspondingly threadingly engage threaded portions 41, 42 of central rod member 40 upon assembly of vertebral body replacement device 10. Although not shown in FIGS. 2A and 2B, internal wall 23 also includes at least one channel 25 (see FIG. 1) with at least one channel 25 being oriented substantially vertical and is sized to receive corresponding at least one anti-rotational rib 35 of body member 30 when vertebral body replacement device 10 is assembled.

As further shown in the cross-sectional views of FIGS. 2A and 2B, end member 20 has an external wall 22, through which at least one hole 27 passes to adjacent internal wall 23. At least one hole 27 is sized to allow for the placement of bone graft material and other biocompatible materials for the purpose of facilitating a bone fusion bed following implantation.

Additionally, as seen in FIGS. 1 and 2A, end wall 24 functions to cap or bound inner portion 21 at one end of end member 20. End wall 24 is integrally coupled to threaded housing element 28 and generally includes at least one projection 26 or engagement element that extends in an outward direction from the outer surface of end wall 24. At least one projection 26 may be configured as a tooth-like body (as shown in FIGS. 1, 2A, 2B, and 5) although other shaped projections or engagement elements are contemplated including, but not limited to spikes, pegs, grids, fingers and posts. At least one projection 26 is sized to allow for operative engagement with the adjacent vertebral body, more specifically with the anatomic end plate of the vertebral body to provide adequate fixation post-implantation and to withstand any torsional loads that may be applied to end member 20 following implantation and during the lengthening procedure of vertebral body replacement device 10.

Cross-section view of FIG. 2A shows, end wall 24 being oriented perpendicular or normal relative to external wall 22. FIG. 2B shows an alternative embodiment of end member 20 with end wall 24 being oriented at an angle α and relative to external wall 22. Having end wall 24 being angled provides the operating surgeon with the ability to treat clinically, lordotic and kyphotic deformities. It should be well understood to those skilled in the art that end member 20 will be offered in a wide range of degrees of angulations in varying increments from 0° to 20°, thereby providing the operating surgeon with the ability to precisely treat any deformity presented during a surgical procedure.

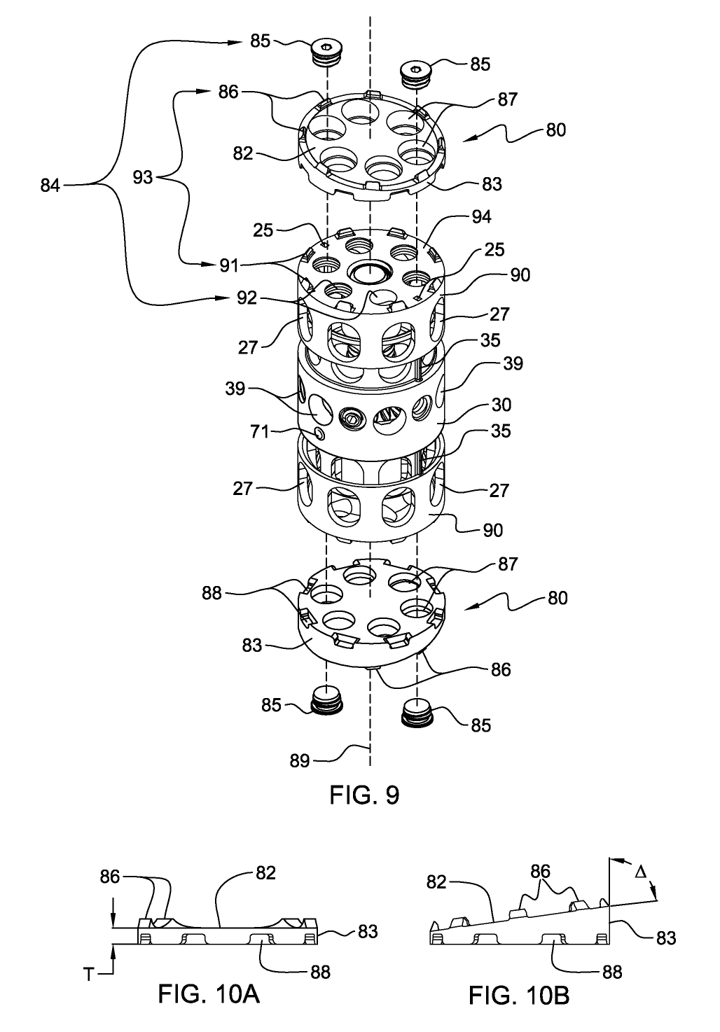

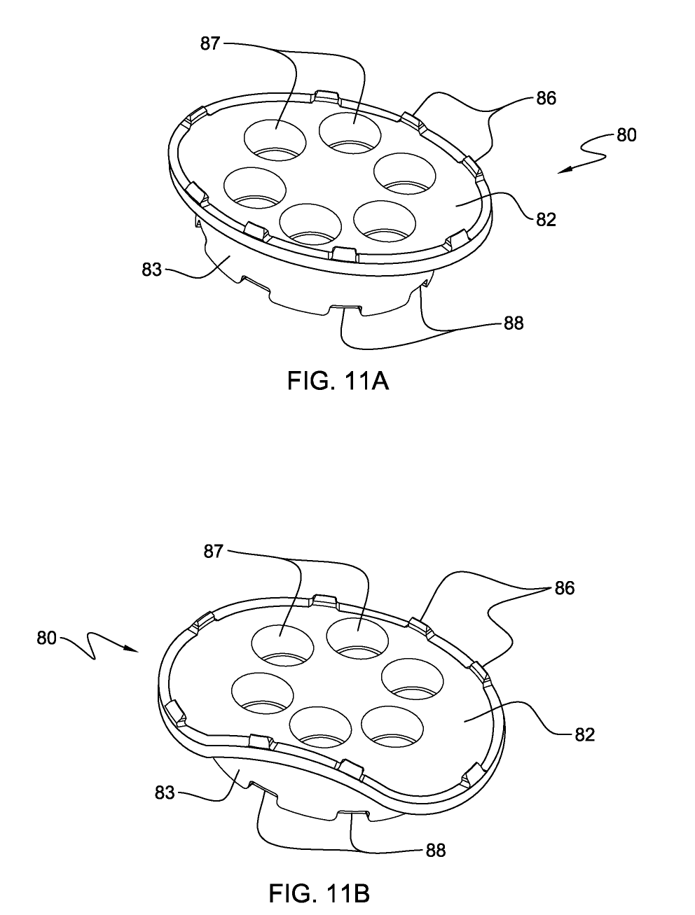

As shown in FIG. 9, it is contemplated that, vertebral body replacement device 10 may include an alternative embodiment of end member 90, with end wall 94 being configured to couple a footplate member 80. End wall 94 may further include at least one alignment tab 91 that functions to orient footplate member 80 in the preferred position relative to a central axis 89 and end member 90, and a vertebral body following implantation. As seen in FIGS. 11A and 11B, it is contemplated that footplate member 80 will be available in a plurality of various circular, non-circular and polygonal outer profile shapes, (i.e., circular as shown in FIG. 9, oval as shown in FIG. 11A, kidney as shown in FIG. 11B or oblong (not shown)) and sizes. It is further contemplated that footplate member 80 will be available in varying thicknesses or heights T as seen in FIG. 10A. Having a kit or implant system that includes a range of various sized heights, shapes, sizes and angled footplate members 80 provides the operating surgeon with multiple choices to maximum bone coverage, spine alignment and resulting stability of the device relative to the adjacent vertebral body following implantation.

As shown in FIG. 10A, an end surface 82 may be configured in a neutral or normal orientation relative to a sidewall or mating surface 83 of footplate member 80. Alternatively, FIG. 10B shows footplate member 80 having end surface 82 being angled (angle Δ) relative to sidewall or mating surface 83. As discussed above, it is contemplated that the operating surgeon will be provided with a plurality of footplate members 80 each having a different angle, with angulation ranging from 0° to 20°. Having such a wide range of incrementally angled footplate members 80 available will provide the operating surgeon with the ability to customize the vertebral body replacement device 10 during the operative procedure to meet the presented clinical deformity. Although shown with a circular perimeter geometry in FIG. 9, as described previously it should be understood to those skilled in the art that both neutral and angled footplate members 80 will be constructed in multiple outer profile geometric shapes, sizes and overall thickness T, again to provide the operating surgeon with the ability to maximize bone support post-implantation. Footplate member 80 may be modular in design, thereby allowing the operating surgeon to mix and match and interchange footplate members 80 with end member 90. This is accomplished by securely attaching and allowing detachment of footplate member 80 from end wall 94 of end member 90 by use of a locking mechanism 84. For example purposes only, as shown, locking mechanism 84 may consist of at least one locking screw 85 that passes through a hole 87 in end surface 82 to engage corresponding threaded holes 92 in end wall 94. Further, it should be understood to those skilled in the art that various other low-profile locking or securement mechanisms may also be used for this purpose including, but not limited to lock pins, bolts, and press fit pins.

As described above, it is contemplated that footplate member 80 will also include at least one projection 86 or engagement element that extends outwardly from the end surface 82. At least one projection 86 may be configured as a tooth-like projection (as shown in FIGS. 9, 10A, and 10B) although other shaped engagement elements are contemplated, including but not limited to, spikes, pegs, grids, figures and posts. End surface 82 may be treated or coated with certain materials to facilitate bio-ingrowth with the adjacent vertebral body following implantation. Additionally, end surface 82 may also undergo a process or treatment that results in end surface 82 having nano-sized or micron-sized surface features.

As seen in FIG. 9, footplate member 80 has an orientation or adjustment mechanism 93 that may include alignment slots 88 that are positioned along mating surface or sidewall 83. Slots 88 will slidingly engage corresponding tabs 91 positioned around the peripheral of end member 90. Orientation or adjustment mechanism 93 functions to facilitate the positioning of footplate member 80 relative to end member 90 more specifically to end wall 94 and ultimately when implanting, the adjacent vertebral body within the spinal column of a patient. Footplate member 80 when moved is typically rotated or moved relative to central axis 89 and end member 90. It is contemplated further that an alternative embodiment of orientation or adjustment mechanism 93 may be positioned in the more central portions of end wall 94 and end surface 82, respectively.

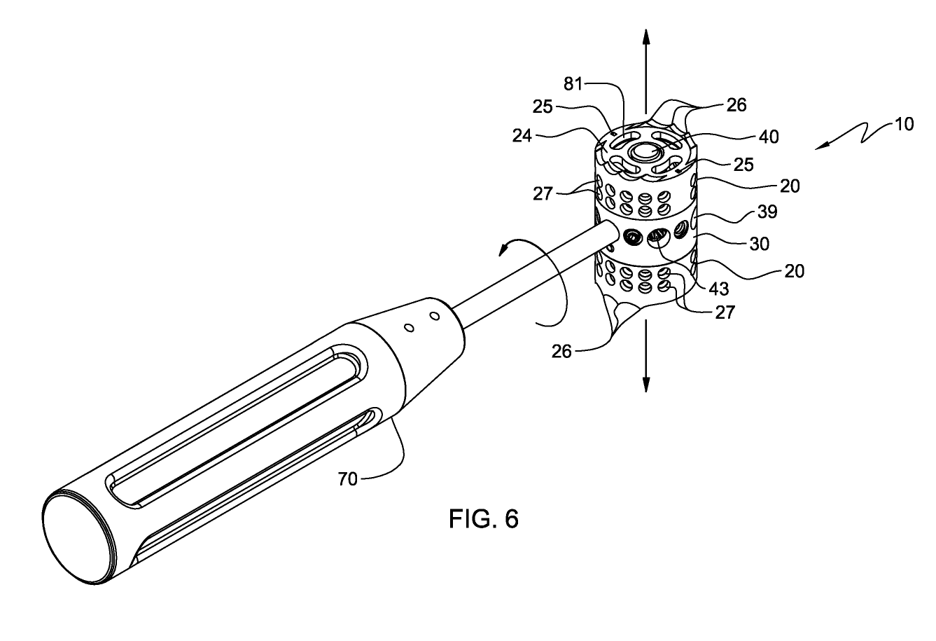

Following the assembly of vertebral body replacement device 10, superiorly positioned or first end member 20 and inferiorly positioned or second end member 20 are both positioned with each respective inner portion 21 and threaded housing element 28 within first end receptacle 33 and second end receptacle 34, respectively. As shown in FIG. 6, first end member 20 and second end member 20 may be simultaneously extended or retracted in an axial direction relative to body member 30 resulting in either the lengthening or shortening of the over-all length of vertebral body replacement device 10 by inserting a tool 70 through tool port hole 39 to engage the gear shaped tip (not shown) of tool 70 with tooth faced surface 44 of gear wheel portion 43 of central rod member 40. In operation, tool 70 is rotated causing gear wheel portion 43 to rotate resulting in first and second threaded portions 41, 42 rotating about central axis 46. When assembled, threaded housing element 28 of first and second end members 20 are threaded onto first and second threaded portions 41, 42 of central rod member 40 respectively, with at least one channel 25 of first and second end members 20 also engaging at least one anti-rotational rib 35 positioned on outer wall 32 of first and second end receptacles 33, 34, respectively. Functionally, the engagement of at least one channel 25 of first and second end members 20 with at least one rib 35 of body member 30 prohibits rotational movement of the first and second end members 20 when tool 70 is turned, thus resulting in first and second end members 20 simultaneously advancing or moving in opposing axial directions relative to body member 30 for a maximum distance equal to the thread length of first and second thread portions 41, 42 of central rod member 40. As discussed above, the bi-directional axial motion of the first end and second end members 20 is caused by the opposing threads (i.e., right-handed and left handed threads) of the respective first and second threaded portion 41, 42 of the central rod member 40. Operationally, central rod member 40 converts the rotational motion of tool 70 and gear wheel portion 43 into corresponding axial or linear movement of first and second end members 20, with the mating of channel 25 and rib 35 substantially prohibiting any rotational movement of two end members 20 relative to longitudinal axis 72 and the adjacent vertebrae, thus eliminating torsional forces being applied to the end member-vertebral body interface. For example purposes, FIG. 5 shows an assembled vertebral body replacement device 10 following partial simultaneous movement of first and second end members 20 as describe above.

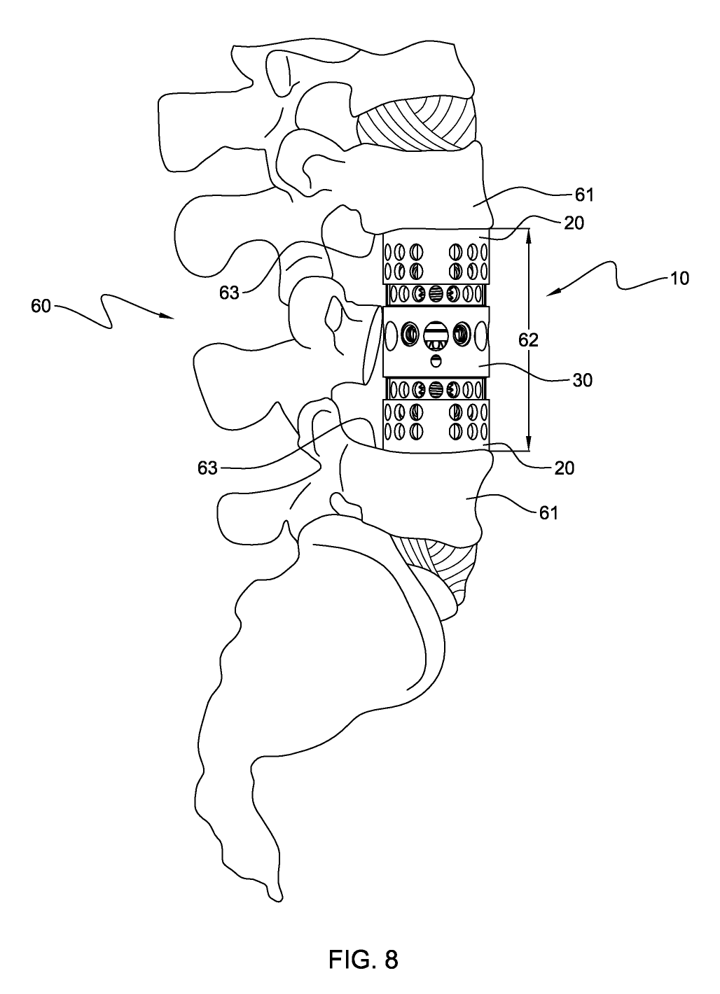

FIG. 8 shows assembled vertebral body replacement device 10 positioned within a space between two vertebral bodies following simultaneous movement of first and second end members 20 in the manner described above, resulting in intimate contact between an adjacent vertebral body and at least one projection 26 extending from end wall 24, or alternatively, projection 86 of footplate member 80 (not shown). A resultant compressive force is applied by each end member 20 (or footplate member 80) against the contacted vertebral body to maintain the desired anatomic spacing.

The surgical technique for implantation of a vertebral body replacement device is well known in the art, including the appropriate surgical exposure and dissection techniques. The method includes, obtaining a vertebral body replacement device 10 that may include body member 30, central rod member 40 that has two threaded portions 41, 42 and is configured to be operatively associated within body member 30 and first and second end members 20 that are configured to threadingly engage the two threaded portions 41, 42 of central rod member 40. As discussed above, body member 30 and end members 20 are further configured to inhibit rotational movement of two end members 20 following assembly and positioning of vertebral body replacement device 10 within a space within a spinal column with both end members 20 engaging respective vertebral bodies when central rod member 40 is rotationally actuated, thus causing two end members 20 to move in opposing axial directions relative to body member 30. Upon such movement, two end members 20 will apply a force to the two adjacent vertebral bodies within the spinal column. It should be understood that all of the above noted device components and respective elements include the same structural and functionality characteristics as described previously herein.

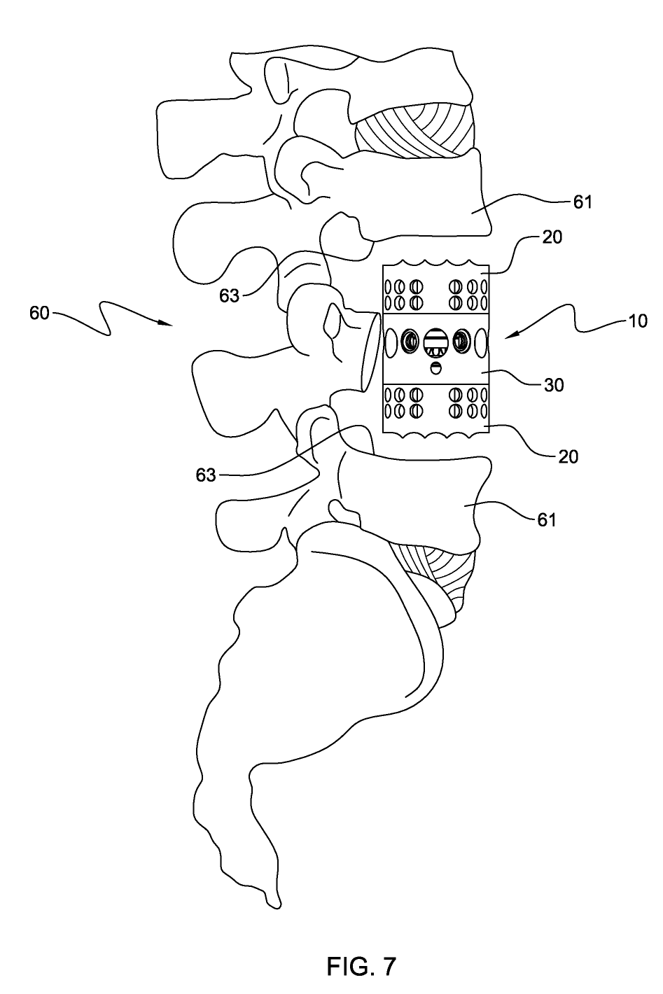

As seen in FIG. 7, the method may further include the step of positioning vertebral body replacement device 10 between two vertebral bodies within a patient's spinal column. The surgical method may also include the step of simultaneously operatively moving in opposing directions both end members 20 relative to body member 30 to produce a force against the two respective adjacent vertebral bodies for the purpose of maintaining a space between the two vertebral bodies within the spinal column as shown in FIG. 8. Although not shown, the method may further include the step of engaging tool 70 with central rod member 40 through tool portal hole 39, whereby rotary motion of tool 70 is converted into opposing axial movement of two respective end members 20 relative to body member 30 causing two end members 20 to come in contact and apply a force to the adjacent vertebral bodies, thereby maintaining the space between these two vertebral bodies. The method also may include the step of securely coupling to body member 30 a lock pin through lock pin hole 71 following finalization of the length adjustment procedure to ensure securement of two end members 20 relative to body member 30 and central rod member 40.

It should be understood by those skilled in the art that the surgical method described herein may also include, alternatively, using modular footplate member 80 that has been coupled to alternative embodiment end member 90 which has been more fully described above. The sequence of implantation of vertebral body replacement device 10 as described herein may be different depending upon the given clinical situation and whether footplate members 80 are attached on the “back table” prior to the complete assembly of vertebral body replacement device 10 or within the operative site. It is contemplated that footplate member 80 would be oriented relative to end member 80 and a vertebral body within the spine to facilitate and maximize bone contact and stability. Final securement and positioning of footplate member 80 to end member 80 is dependent upon the operating surgeon achieving adequate alignment during trial implantation. Once this has been achieved, orientation or adjustment mechanism 93 will be used to finalize the position of footplate member 80 relative to the vertebral body, with locking mechanism 84 being actuated to secure footplate member 80 to end member 90. The sequence of device orientation, assembly and securement will be at the discretion of the operating surgeon and will vary depending upon the preference of the operating surgeon in combination with the clinical needs of the patient.

It is further contemplated that an implant system comprised of various cross-sectional sizes, cross-sectional polygonal and circular/oval shapes and longitudinal lengths of body members 30, end members and footplate member 80 will be available as a kit. This will allow the operating surgeon to pick and choose the separate member components to assemble vertebral body replacement device 10 that best fits into a certain spinal segment or to address an anatomical deformity presented in a patient. It should be understood by those skilled in the art that each shaped and dimensioned member provided will function in the same manner as described previously herein with central rod member 40 and supporting ring 50.

In one example, a method for assembling vertebral body replacement device 10 includes obtaining body member 30 that is an elongate body having inner wall 31 and outer wall 32 and includes first end receptacle 33, second end receptacle 34 and longitudinal axis 72 extending between first end receptacle 33 and second end receptacle 34. The method also includes obtaining central rod member 40 that has first threaded portion 41, second threaded portion 42 and central axis 46 extending therebetween. Central rod member 40 is configured to be operatively associated with body member 30. The method includes obtaining first end member 20 and second end member 20 with first end member 20 being configured to be positioned within first end receptacle 33 of body member 30 to threadingly engage first threaded portion 41 of central rod member 40 when central rod member 40 is operatively associated with body member 30 and second end member 20 being configured to be positioned within second end receptacle 34 of body member 30 to threadingly engage second threaded portion 42 of central rod member 40 when central rod member 40 is operatively associated with body member 30. The further method includes obtaining at least one footplate member 80 which has end surface 82 with end surface 82 being configured to engage a bone surface upon implantation of the vertebral body replacement device. At least one footplate member 80 also has a sidewall adjustment or mating surface 83 attached to end surface 82 with sidewall 83 being configured to have orientation adjustment mechanism 93. The orientation adjustment mechanism 93 functions to align at least one footplate member 80 in a certain position relative to first end member 20 and/or the second end member 20 and a vertebral body. At least one footplate member 80 also has locking mechanism 84 that is configured to securely couple at least one footplate member 80 to the first end member 20 and/or the second end member 20, thus allowing the at least one footplate member 80 to be modular in function. The method also includes placing central rod member 40 within middle chamber 36 of the body member 30 and operatively associating central rod member 40 with body member 30. The method may include threadingly engaging first threaded portion 41 of central rod member 40 with first end member 20 and threadingly engaging second threaded portion 42 of central rod member 40 with the second end member 20. An additional step of the method may also include employing locking mechanism 84 to connect at least one footplate member 80 to first end member 20 and/or second end member 20.

In another example, a method of using at least one footplate 80 member in a vertebral body replacement device 10 includes obtaining vertebral body replacement device 10 that includes body member 30, central rod member 40 including first threaded portion 41 and second threaded portion 42. Central rod member 40 is configured to be operatively associated within body member 30 and first end member 20 and second end member 20. First end member 20 is configured to threadingly engage first threaded portion 41 of central rod member 40 and second end member 20 is configured to threadingly engage second threaded portion 42 of central rod member 40. The method also includes obtaining at least one footplate member 80 or mating surface 83 that has end surface 82 and sidewall or mating surface 83 that is attached to end surface 82. Sidewall 83 is configured to have orientation adjustment mechanism 93 which functions to align the at least one footplate member 80 in a certain position relative to first end member 20 and/or second end member 20. At least one footplate member 80 also has locking mechanism 84 that is configured to securely couple the at least one footplate member 80 to first end member 20 and/or second end member 20. The method further includes coupling at least one footplate member 80 to first end member 20 and/or second end member 20 with locking mechanism 84 securely connecting the at least one footplate member 80 to first end member 20 and/or second end member 20.

Although the preferred embodiments have been depicted and described in detail herein, it will be apparent to those skilled in the relevant art that various modifications, additions and substitutions can be made without departing from its essence and therefore these are to be considered to be within the scope of the following claims.

What is claimed is:

1. A footplate member for use in a vertebral body replacement device, the footplate member comprising:
an end surface, wherein the end surface is configured to engage a bone surface upon implantation of the vertebral body replacement device within a spinal column;
a mating surface connected to the end surface with the mating surface being configured to have an adjustment mechanism, wherein the adjustment mechanism facilitates the positioning of the footplate member relative to an end member of the vertebral body replacement device; and
a locking mechanism, wherein the locking mechanism is configured to couple the footplate member to the end member prior to implantation of the vertebral body replacement device within the spinal column.

2. The footplate member of claim 1, wherein the locking mechanism comprises a set screw and an opening, wherein the set screw is configured to pass through a hole within the footplate member and to be received into an opening disposed within end member, the set screw when threadingly engaged within the opening securely couples the footplate member to the end member.

3. The footplate member of claim 1, wherein the adjustment mechanism comprises a plurality of tabs and corresponding alignment slots, the plurality of tabs being disposed on the end member and the corresponding alignment slots being positioned along the mating surface of the footplate member, wherein each alignment slot is sized to mate with one of the plurality of tabs and, thereby orient the footplate member in a certain position relative to the end member.

4. The footplate member of claim 1, wherein the end surface comprises a plurality of projections, each of the plurality of projections are configured to extend away from the end surface, thereby to engage and secure the footplate member against an adjacent vertebral body upon implantation of the vertebral body replacement device.

5. The footplate member of claim 1, wherein the end surface of the footplate member is oriented substantially normal or at an angle relative to the mating surface.

6. The footplate member of claim 1, wherein an outer profile of the end surface is at least one of a polygonal shape, a non-circular shape and a circular shape, wherein the shape of the outer profile of the end surface is selected to correspond the shape of the adjacent vertebral body following implantation of the vertebral body replacement device within the spinal column.

7. The footplate member of claim 1, wherein the end surface of the footplate member comprises a plurality of surface features, wherein the surface features are micron-sized or nano-sized surface features.

8. The footplate member of claim 1, wherein the end surface of the footplate member is configured and coated with a substance to facilitate bio-ingrowth of the end surface to an adjacent vertebral body following implantation of the vertebral body replacement device within a spinal column.

9. A method for assembling a vertebral body replacement device, the method comprising:
obtaining a body member, wherein the body member is an elongate body having an inner wall and an outer wall, and comprising a first end receptacle, a second end receptacle and a longitudinal axis extending between the first end receptacle and the second end receptacle thereof;
obtaining a central rod member having a first threaded portion, a second threaded portion and a central axis extending therebetween, the central rod member being configured to be operatively associated with the body member;
obtaining a first end member and a second end member, wherein the first end member is configured to be positioned within the first end receptacle of the body member to threadingly engage the first threaded portion of the central rod member when the central rod member is operatively associated with the body member, and the second end member is configured to be positioned within the second end receptacle of the body member to threadingly engage the second threaded portion of the central rod member when the central rod member is operatively associated with the body member; and
obtaining at least one footplate member, wherein the at least one footplate member comprises:
an end surface, wherein the end surface is configured to engage a bone surface upon implantation of the vertebral body replacement device within a spinal column;
a mating surface connected to the end surface with the mating surface being configured to have an adjustment mechanism, wherein the adjustment mechanism facilitates the positioning of the at least one footplate member relative to at least one of the first end member and second end member; and
a locking mechanism, wherein the locking mechanism is configured to couple the at least one footplate member to at least one of the first end member and second end member, thereby allowing the footplate to be modular;
placing the central rod member within a middle chamber of the body member and operatively associating the central rod member with the body member;
threadingly engaging the first threaded portion of the central rod member with the first end member and threadingly engaging the second threaded portion of the central rod member with the second end member;
employing the locking mechanism to connect at least one footplate member to at least one of the first end member and second end member prior to implanting the vertebral body replacement device within a spinal column.

10. The method of claim 9, wherein the obtaining at least one footplate member further comprises the adjustment mechanism comprising a plurality of tabs and corresponding alignment slots, the plurality of tabs being disposed on an end wall of each of the first end member and the second end member and the corresponding alignment slots being positioned along the mating surface of the footplate member, wherein each alignment slot is sized to mate with one of the plurality of tabs and, thereby orient the footplate member in a certain position relative to the end wall of at least one of the first end member and second end member and a vertebral body following implantation of the vertebral body replacement device.

11. The method of claim 9, wherein the end surface of the at least one footplate member is oriented substantially normal or at an angle relative to the mating surface.

12. The method of claim 9, wherein an outer profile of the end surface of the at least one footplate member is at least one of a polygonal shape, a non-circular shape a circular shape, wherein the shape of the outer profile of the end surface is selected to correspond the shape of the adjacent vertebral body following implantation of the vertebral body replacement device within a spinal column.

13. The method of claim 9, wherein the end surface of the at least one footplate member comprises a plurality of surface features with the surface features being micron-sized or nano-sized.

14. The method of claim 9, wherein the end surface of the at least one footplate member is configured and coated with a substance to facilitate bio-ingrowth of the end surface to an adjacent vertebral body following implantation of the vertebral body replacement device within a spinal column.

15. The method of claim 9, further comprising obtaining a support ring, wherein the support ring has a bearing surface, and is configured to threadingly engage the inner wall of the body member, thereby allowing the bearing surface to contact the central rod member when the central rod member is operatively positioned within the body member.

16. The method of claim 15, wherein the obtaining a central rod member further comprises a gear wheel portion, the gear wheel portion having a toothed face surface and a support surface, the support surface of the gear wheel portion being configured to contact the bearing surface of the support ring when the central rod member is operatively positioned within the body member.

17. The method of claim 16, wherein the central rod member comprises a central axis extending between the first threaded portion and the second threaded portion thereof, and wherein the rotational axis of the gear wheel portion is substantially coaxial to the central axis of the central rod member, thereby when the gear wheel portion is rotated about the rotational axis the first and second threaded portions correspondingly rotate about the central axis of the central rod member.

18. A method of using at least one footplate member in a vertebral body replacement device, the method comprising:
obtaining a vertebral body replacement device, the vertebral body replacement device including a body member, a central rod member having a first threaded portion and a second threaded portion, the central rod member being configured to be operatively associated within the body member and a first end member and a second end member, the first end member being configured to threadingly engage the first threaded portion of the central rod member and the second end member being configured to threadingly engage the second threaded portion of the central rod member;
obtaining at least one footplate member, the at least one footplate member comprising an end surface, a mating surface connected to the end surface with the mating surface being configured to have an adjustment mechanism, wherein the adjustment mechanism facilitates the positioning of the at least one footplate member relative to at least one of the first end member and second end member, and a locking mechanism, wherein the locking mechanism is configured to couple the at least one footplate member to at least one of the first end member and second end member; and
coupling the at least one footplate member to at least one of the first end member and second end member, the locking mechanism securely connecting the at least one footplate member to at least one of the first end member and second end member.

19. The method of claim 18, further comprising surgically locating and exposing a space within a spinal column of a patient.

20. The method of claim 19, further comprising determining the size of the exposed space within the spinal column of the patient.

21. The method of claim 20, further comprising selecting a vertebral body replacement device that corresponds in size to the exposed space within the spinal column.

22. The method of claim 21, further comprising inserting the vertebral body replacement device and coupled at least one footplate member into the exposed space within the spinal column.

23. The method of claim 24, further comprising rotationally actuating the central rod member to move in an axial direction relative to the body member the at least one footplate member coupled to at least one of the first end member and the second end member, thereby resulting in the at least one footplate member applying a force to two vertebral bodies to maintain the space within the spinal column of a patient.
